SEQUENCE LISTING (3 sequences)

SEQ ID NO: 1 - protein, 571 aa, Artificial
  Artificial polypeptide
GAPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPG

SEQ ID NO: 2 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 3 - protein, 189 aa, Artificial
  Artificial Polypeptide
GAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPP

(19) United States
(12) Patent Application Publication
(10) Pub. No.: US 2020/0296940 A1
(43) Pub. Date: Sep. 24, 2020

(54) PEARL CULTURE MATERIAL AND COATING COMPOSITION

(71) Applicant: FUJIFILM CORPORATION, Tokyo (JP)

(72) Inventors: Akihito AMAO, Kanagawa (JP); Kazutaka CHIBANA, Kanagawa (JP); Hidehiro MOCHIZUKI, Kanagawa (JP); Tadanori YAMADA, Kanagawa (JP)

(21) Appl. No.: 16/862,590

(22) Filed: Apr. 30, 2020

Related U.S. Application Data
(63) Continuation of application No. PCT/JP2018/036741, filed on Oct. 1, 2018.

(30) Foreign Application Priority Data
Nov. 6, 2017 (JP) 2017-214180

(51) Int. Cl. A01K 61/57; C09D 189/00; C07K 14/78; A01K 61/56

(52) U.S. Cl. CPC A01K 61/57; A01K 61/56; C07K 14/78; C09D 189/00

(57) ABSTRACT

Provided are a pearl culture material in which at least one selected from the group consisting of a pearl nucleus and a mantle piece is coated with a protein containing a repeating sequence of GXY triplets which may be separated by one or more amino acids, and one or more RGD motifs, and having a polydispersity of less than 20; and a coating composition including the protein.

### CROSS-REFERENCE TO RELATED APPLICATIONS

This application is a continuation application of International Application No. PCT/JP2018/036741, filed Oct. 1, 2018, the disclosure of which is incorporated herein by reference in its entirety. Further, this application claims priority from Japanese Patent Application No. 2017-214180, filed Nov. 6, 2017, the disclosure of which is incorporated herein by reference in its entirety.

### INCORPORATION OF SEQUENCE LISTING

The Sequence Listing associated with this application is provided in text format as a file entitled “FS-F07913-01_SequenceListing.TXT”, created on Mar. 4, 2020. The text file, filed via EFS-Web, has a size of 20 kb. The information in electronic format of the Sequence Listing is incorporated herein by reference in its entirety.

### BACKGROUND OF THE INVENTION

### 1. Field of the Invention

The present disclosure relates to a pearl culture material and a coating composition.

### 2. Description of the Related Art

Pearls can be produced by inserting a small piece of mantle and a pearl nucleus into a body of a mother pearl oyster which is capable of producing pearls, and allowing to secrete a nacreous substance on a surface of the pearl nucleus. Various techniques have been proposed for the purpose of obtaining a high quality pearl.

For example, there has been disclosed a mother nucleus for cultured pearls, characterized in that a surface of a sphere mainly composed of plastic having a specific gravity of 2.3 to 2.7 and having a small amount of eluted material is coated with a film mainly composed of insolubilized collagen (see, for example, JP1975-075997U (JP-550-075997U)).

In addition, there has been disclosed a nucleus for pearl culture, characterized in that the surface of a nucleus for culture is coated with a cell-scaffolding organic substance that is nontoxic to pearl oysters and is less rejective than calcium crystals (see, for example, JP1989-148135A (JP-H01-148135A)).

In addition, as a cell activator for pearl culture which is used for treating a mantle section of a mother pearl oyster for pearl culture, there has been disclosed a mantle cell activator for pearl culture, characterized by containing an artificial polypeptide which has an Arg-Gly-Asp sequence in a molecule thereof and has a cell adhesion activity and a cell activation activity (see, for example, JP1993-236848A (JP-H05-236848A)).

In addition, there has been disclosed a method for treating a pearl nucleus, characterized by treating a mantle section used for an inserted nucleus with naturally occurring vitronectin (see, for example, JP1995-132032A (JP-H07-132032A)).

In addition, there has been disclosed a pearl nucleus characterized in that the surface of the nucleus has a positive charge (see, for example, WO2015/033972A).

In addition, there has been disclosed a method for culturing pearls in which a piece obtained from the mantle of a piece shellfish and a pearl nucleus are transplanted into a mother pearl oyster to culture pearls, characterized by separating epithelial cells from an epithelial tissue of the mantle as the piece, and using the separated epithelial cells artificially cultured in vitro (see, for example, JP2006-304628A).

### SUMMARY OF THE INVENTION

However, irrespective of the existence of JP1975-075997U (JP-S50-075997U), JP1989-148135A (JP-H01-148135A), JP1993-236848A (JP-H05-236848A), JP1995-132032A (JP-H07-132032A), WO2015/033972A, and JP2006-304628A, a yield of a high quality pearl cannot be said to be sufficiently high, and there is still a problem of improving the yield of the high quality pearl.

In addition, in a case of producing pearls, it is necessary to insert a mantle piece (a small piece of the mantle) and a pearl nucleus into the body of a mother pearl oyster which is capable of producing pearls (nucleus insertion), but a protein that coats the pearl nucleus or mantle piece, described in JP1975-075997U (JP-550-075997U), JP1989-148135A (JP-H01-148135A), JP1993-236848A (JP-H05-236848A), JP1995-132032A (JP-H07-132032A), and WO2015/033972A, has a problem in that, since the protein has high adhesiveness, the operability at the time of nucleus insertion into the mother pearl oyster is poor and therefore the efficiency of the nucleus insertion operation is reduced.

In view of the above, an object to be achieved by one embodiment of the present invention is to provide a pearl culture material that improves a yield of a high quality pearl and is excellent in the operability of nucleus insertion into a mother pearl oyster and a coating composition for producing a pearl culture material.

Specific means for achieving the foregoing object include the following aspects.
<1> A pearl culture material in which at least one selected from the group consisting of a pearl nucleus and a mantle piece is coated with a protein containing a repeating sequence of GXY triplets which may be separated by one or more amino acids, and one or more RGD motifs, and having a polydispersity of less than 20.
<2> The pearl culture material according to <1>, in which the protein has a weight-average molecular weight of 30 kDa to 200 kDa determined by gel permeation chromatography.
<3> The pearl culture material according to <1> or <2>, in which the protein comprises at least a part of an amino acid sequence of collagen.
<4> The pearl culture material according to <3>, in which the amino acid sequence of the collagen is an amino acid sequence of type I collagen α1 chain.
<5> A coating composition for coating at least one selected from a pearl nucleus or a mantle piece, comprising:
a protein containing a repeating sequence of GXY triplets which may be separated by one or more amino acids, and one or more RGD motifs, and having a polydispersity of less than 20.

According to the present disclosure, there are provided a pearl culture material that improves a yield of a high quality pearl and is excellent in the operability of nucleus insertion into a mother pearl oyster and a coating composition for producing a pearl culture material.

### DESCRIPTION OF THE PREFERRED EMBODIMENTS

In the present disclosure, a numerical range indicated using “to” means a range including numerical values described before and after “to” as a minimum value and a maximum value, respectively. In a numerical range described in a stepwise manner in the present disclosure, an upper limit value or a lower limit value described in a certain numerical range may be replaced with an upper limit value or a lower limit value of another numerical range described in a stepwise manner. In addition, in the numerical range described in the present disclosure, the upper limit value or the lower limit value described in a certain numerical range may be replaced with the values shown in the Examples.

In the present disclosure, in a case where a plurality of substances corresponding to components are present in the composition, the amount of each component in the composition means a total amount of the plurality of substances present in the composition, unless otherwise noted.

In the present disclosure, the term “peptide” refers to a generic term for compounds formed by binding two or more amino acids through peptide bonds.

In the present disclosure, the term “polypeptide” refers to a generic term for compounds formed by peptide bonding of 10 or more amino acids. In a case where the number of amino acids is 10 or more, the “peptide” and the “polypeptide” can be used interchangeably.

In the present disclosure, the term “protein” refers to a polypeptide having a molecular weight of 5000 or more. In a case where the molecular weight is 5000 or more, the “protein” and the “polypeptide” can be used interchangeably.

In the present disclosure, the weight-average molecular weight (Mw) and the number-average molecular weight (Mn) of a protein are expressed in units of dalton (Da).

In the present disclosure, the term “pearl nucleus” refers to a material in which a pearl layer made of a nacreous substance mainly composed of calcium carbonate secreted from epithelial cells of the mantle is formed on the surface thereof by being inserted into a pearl oyster together with a mantle piece. The size of the pearl nucleus may be appropriately selected according to a desired size of the pearl, and the pearl nucleus is, for example, a spherical material of 4 mm to 10 mm. The material for the pearl nucleus is typically a shell material of a freshwater bivalve, and examples of the freshwater bivalve include the genus Lamprotula, pig-toe mussel (Fusconaia flava), and niggerhead mussel (Fusconaia ebenus).

In the present disclosure, the term “mantle piece” refers to a small piece of mantle of a shellfish which is inserted into a pearl oyster together with a pearl nucleus to thereby secrete a nacreous substance mainly composed of calcium carbonate from the epithelial cells of the mantle contained in the mantle piece, thus resulting in the formation of a pearl layer on the surface of the pearl nucleus. Shellfish used for producing the mantle piece include Pinctada fucata martensii and the like. The size of the mantle piece may be appropriately selected according to the size of the pearl nucleus and is, for example, about 2 mm to 4 mm square.

In the present disclosure, the weight-average molecular weight and the number-average molecular weight of a protein are values determined as pullulan-converted molecular weights measured by gel permeation chromatography using a GPC apparatus (HLC-8220GPC, manufactured by Tosoh Corporation) under the following conditions. An aqueous solution of a protein to be measured (after thawing for a frozen preparation) is heated at 40° C. for 30 minutes to completely dissolve the protein, diluted with 100 mM phosphate buffer such that the concentration of the protein to be measured is 0.2% by mass, and then filtered through a 0.45 μm filter to prepare a sample solution. This sample solution is measured using the above-mentioned GPC apparatus to obtain a weight-average molecular weight and a number-average molecular weight.

[GPC Measurement Conditions]

Column: Shodex Asahipak GS-620 7G-P, inner diameter: 7.5 mm, length: 50 cm (manufactured by Showa Denko K.K.)

Eluent: 100 mM phosphate buffer (pH=6.9)

Flow rate: sample: 1.0 mL/min, reference: 0.1 mL/min

Column temperature (setting): 40° C.

Injection volume: 100 μL

Detector: RI/UV (210 nm)

Protein for molecular weight calibration: pullulan (Shodex P-82, manufactured by Showa Denko K.K.)

In the present disclosure, the term “tackiness” means “adhesiveness” or “stickiness” used in relation to the workability in a case of carrying out insertion of a pearl culture material.

<Pearl Culture Material>

In the pearl culture material of the present disclosure, at least one selected from the group consisting of a pearl nucleus and a mantle piece is coated with a protein containing a repeating sequence of GXY triplets which may be separated by one or more amino acids, and one or more RGD motifs, and having a polydispersity of less than 20.

Pearls can be produced by inserting a mantle piece and a pearl nucleus into a body of a mother pearl oyster which is capable of producing pearls, and allowing to secrete a nacreous substance on a surface of the nucleus. Various techniques have been proposed for the purpose of obtaining a high quality pearl, but the yield of the high quality pearl is not sufficiently high.

In addition, in a case of producing pearls, it is necessary to insert the mantle piece and the pearl nucleus into the body of a mother pearl oyster which is capable of producing pearls. In this regard, although the related art discloses mantle pieces or pearl nuclei coated with specific proteins or polypeptides, these proteins or polypeptides that coat the mantle pieces or pearl nuclei have high adhesiveness, which results in poor operability at the time of nucleus insertion into a mother pearl oyster and therefore reduced working efficiency.

On the other hand, according to the present disclosure, it is possible to provide a pearl culture material that improves a yield of a high quality pearl and is excellent in the operability of nucleus insertion into a mother pearl oyster. The reason for providing such an effect is not clear, but it is speculated as follows. That is, it is considered that the protein of the present disclosure, which coats at least one selected from the group consisting of a pearl nucleus and a mantle piece, contains one or more RGD motifs involved in cell adhesion, and as a result, the extension of mantle cells to the surface of the pearl nucleus after the nucleus insertion is promoted, and thus a high quality pearl with few stains is generated. In addition, it is considered that the protein of the present disclosure, which coats at least one selected from the group consisting of a pearl nucleus and a mantle piece, contains a repeating sequence of GXY triplets which may be separated by one or more amino acids, thereby having a structure similar to a collagen protein having high biocompatibility, and in a case where at least one selected from the group consisting of a pearl nucleus and a mantle piece, each of which is coated with the protein, is inserted, the yield of a high quality pearl is improved. In addition, it is considered that, since the protein which coats at least one selected from the group consisting of a pearl nucleus and a mantle piece has a polydispersity of less than 20 and is a highly unity macromolecule, the interaction between the pearl nucleus and the mantle tissue in a mother pearl oyster at a biological tissue level can be equalized, whereby the effect of improving the yield of a good quality pearl can be exhibited. In addition, it is considered that, since the protein which coats at least one selected from the group consisting of a pearl nucleus and a mantle piece has a polydispersity of less than 20 and is a highly unity macromolecule, the bonds between polar hydrophilic groups between protein molecules are formed preferentially over the bonds due to intermolecular interaction between polar hydrophilic groups and water molecules, and as a result, moisture absorption due to moisture in the air, which is a main cause of tackiness, (that is, the intermolecular interaction with water) is inhibited, and therefore the operability at the time of nucleus insertion is improved.

(Protein)

The protein of the present disclosure contains a repeating sequence of GXY triplets which may be separated by one or more amino acids, and one or more RGD motifs, and has a polydispersity of less than 20.

The weight-average molecular weight of the protein of the present disclosure is preferably 30 kDa (kilodalton) to 200 kDa, more preferably 30 kDa to 100 kDa, still more preferably 40 kDa to 75 kDa, and most preferably 50 kDa to 60 kDa. In a case where the weight-average molecular weight of the protein of the present disclosure is within the above range, deliquescence due to excessive moisture absorption is suppressed while the protein has good solubility in water.

The number-average molecular weight of the protein of the present disclosure is preferably 15 kDa to 100 kDa, more preferably 20 kDa to 80 kDa, still more preferably 30 kDa to 60 kDa, and most preferably 40 kDa to 50 kDa. In a case where the number-average molecular weight of the protein of the present disclosure is within the above range, deliquescence due to excessive moisture absorption is suppressed while the protein has good solubility in water.

—Polydispersity—

In the present disclosure, the polydispersity of a protein is a value (Mw/Mn) obtained by dividing a weight-average molecular weight (Mw) by a number-average molecular weight (Mn).

The protein has a polydispersity of less than 20, preferably a polydispersity of less than 10, more preferably a polydispersity of less than 5, and still more preferably a polydispersity of less than 2. It is considered that, in a case where the polydispersity of the protein of the present disclosure is in the above range, the interaction between the pearl nucleus and the mantle tissue in a mother pearl oyster at a biological tissue level can be equalized, whereby the effect of improving the yield of a good quality pearl can be exhibited. In addition, it is considered that the bonds between polar hydrophilic groups between protein molecules are formed preferentially over the bonds due to intermolecular interaction between polar hydrophilic groups and water molecules, and as a result, moisture absorption due to moisture in the air, which is a main cause of tackiness, is inhibited, and therefore the operability at the time of nucleus insertion is excellent.

—GXY Triplet—

In the present disclosure, the term “GXY triplet” refers to a unit of an amino acid sequence in which three amino acids “G (glycine)”, “X (any amino acid other than G)”, and “Y (any amino acid other than G)” are present from an N-terminal side to a C-terminal side, where “X” and “Y” are each independently any amino acid other than G. “X” or “Y” tends to be “P (proline)” or “4-hydroxyproline”.

The percentage (total percentage) of the region occupied by the GXY triplets to the entire amino acid sequence of the protein of the present disclosure is preferably 50% or more, more preferably 60% or more, still more preferably 70% or more, even more preferably 80% or more, even still more preferably 90% or more, and particularly preferably 95% or more, based on the number of amino acid residues. In addition, the GXY triplet may be repeated over the entire length of the amino acid sequence of the protein of the present disclosure.

Due to the presence of the GXY triplet, the protein of the present disclosure has a structure similar to a collagen protein having high biocompatibility. For this reason, it is considered that the yield of a high quality pearl is improved in a case where at least one selected from the group consisting of a pearl nucleus and a mantle piece, each of which is coated with the protein, is inserted.

In the present disclosure, “GXY triplets” may be connected in series without including an amino acid between the GXY triplets, and one or more amino acids (amino acid residues) may be included between the GXY triplets. Preferably, “GXY triplets” are connected in series without including an amino acid between the GXY triplets.

—RGD Motif—

In the present disclosure, the term “RGD motif” refers to a motif in which three amino acids “R (arginine)”, “G (glycine)”, and “D (aspartic acid)” are present from an N-terminal side to a C-terminal side. It is considered that, since the “RGD motif” is involved in cell adhesion, a pearl culture material having the protein of the present disclosure on the surface of a pearl nucleus or mantle piece is inserted to thereby promote the extension of mantle cells to the surface of the pearl nucleus, resulting in the generation of a high quality pearl with few stains.

The term “motif” used in relation to a protein refers to an amino acid sequence having a functional or structural characteristic.

In the protein of the present disclosure, the number of “RGD motifs” may be 1 to 100, preferably 5 to 75, more preferably 10 to 50, and still more preferably 10 to 25.

In addition, the “RGD motif” is preferably contained at a ratio of 1 to 10 to 100 constituent amino acids of the protein of the present disclosure, more preferably at a ratio of 1 to 20 to 75 constituent amino acids of the protein of the present disclosure, still more preferably at a ratio of 1 to 30 to 60 constituent amino acids of the protein of the present disclosure, and most preferably at a ratio of 1 to 45 to 55 constituent amino acids of the protein of the present disclosure.

In a case where the RGD motif is contained in the protein of the present disclosure in the above range, a cell adhesion promoting ability is in a preferable range, which can contribute to the production of a high quality pearl.

The “GXY triplet” and the “RGD motif” can exist in an overlapping manner. That is, the first G of the GXY triplet may be the second G of the RGD motif.

The protein of the present disclosure preferably includes at least a portion of the amino acid sequence of collagen. More preferably, the protein of the present disclosure includes the amino acid sequence of a collagen domain of collagen (that is, a domain that forms a triple-stranded helix). Here, the collagen domain includes a repeating sequence of GXY triplets which may be separated by one or more amino acids, and one or more RGD motifs.

The percentage (total percentage) of the region occupied by the region derived from the amino acid sequence of collagen (for example, the amino acid sequence of SEQ ID NO: 1) to the entire amino acid sequence of the protein of the present disclosure is preferably 50% or more, more preferably 60% or more, still more preferably 70% or more, even more preferably 80% or more, even still more preferably 90% or more, and particularly preferably 95% or more.

The collagen may be from any species. The collagen is preferably of bovine, porcine, or human origin and more preferably of human origin.

The collagen can be any type of collagen. Examples of the collagen include type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, and type XXVIII collagen.

The amino acid sequence of the collagen, if present, may be an amino acid sequence of any subtype of a plurality of polypeptides constituting the collagen. For example, in a case of type I collagen, the amino acid sequence of the collagen may be the amino acid sequence of type I collagen α1 chain or the amino acid sequence of type I collagen α2 chain; and in a case of type V collagen, the amino acid sequence of the collagen may be the amino acid sequence of type V collagen α1 chain, the amino acid sequence of type V collagen α2 chain, or the amino acid sequence of type V collagen α3 chain.

In the present disclosure, the amino acid sequence of the collagen is preferably the amino acid sequence of type I collagen, and more preferably the amino acid sequence of type I collagen α1 chain.

The protein is preferably a polypeptide including the amino acid sequence of SEQ ID NO: 1 and more preferably a polypeptide made up of the amino acid sequence of SEQ ID NO: 1.

The protein that coats at least one selected from the group consisting of a pearl nucleus and a mantle piece may be a single polypeptide or a combination of two or more polypeptides.

Collagen is contained in cow skin, pig skin, fish skin, or the like, but is insoluble in commonly used solvents such as water. For this reason, in a case where it is attempted to extract collagen, protein hydrolase is used or gelatinization is carried out by heating, which results in the generation of protein molecules having a wide range of molecular weights. For example, commercially available gelatin usually has a molecular weight distribution of tens of thousands to millions. Therefore, even in a case where naturally occurring collagen is extracted by a general method, the polydispersity is not less than 20.

In one embodiment, the protein of the present disclosure can be obtained by subjecting collagen extracted from a natural collagen source, such as cow skin, pig skin, or fish skin, to a fractionation method such as size exclusion chromatography to separate and purify only protein molecules within a specific molecular weight range. In another embodiment, the protein of the present disclosure can be obtained through the production by a recombinant cell into which a gene of a protein containing a repeating sequence of GXY triplets and containing one or more RGD motifs has been introduced. In this case, a bacterial cell such as Escherichia coli, a yeast cell such as S. cerevisiae, or an insect cell such as silkworm can be used as a host cell for introducing the protein gene. As an expression vector, an appropriate vector may be selected from known vectors and used depending on the host and the size of the protein to be introduced. In a case where the protein of the present disclosure is produced by a recombinant cell, a protein having high molecular weight uniformity can be obtained.

The amino acid sequence of human type I collagen α1 chain set forth in SEQ ID NO: 2 contains only two RGD sequences (a sequence of amino acids at positions 745 to 747 and a sequence of amino acids at positions 1093 to 1095) in the total length of 1464 amino acids. In a case where a protein prepared based on the amino acid sequence of human type I collagen α1 chain is used as the protein of the present disclosure, a protein having a more preferable cell adhesion promoting ability is obtained by changing the number of RGD sequences to the preferable range described above. In addition, it is considered that the removal of telopeptides present at both ends of the molecule and contributing to the association between collagen molecules results in the suppression of association between the molecules and the further improved workability at the time of nucleus insertion. In consideration of the above points, the protein of the present disclosure may be, for example, a protein including a plurality of (preferably 4 to 20, and more preferably 6 to 16) regions having a length of 20 amino acids to 60 amino acids including Arg at position 745 to Asp at position 747 in the amino acid sequence of SEQ ID NO: 2. In this case, the plurality of regions each may be the same or different (in other words, the regions around the RGD at positions 745 to 747 overlap but the boundary points are different) and may be in direct contact with each other or one or more other amino acid residues may be interposed therebetween. Each of the regions is preferably a region having a length of 34 amino acids to 50 amino acids including a region of Gly at position 722 to Gly at position 755 independently of each other. In addition, the total amino acid length is preferably 200 amino acids to 800 amino acids and more preferably 300 amino acids to 600 amino acids, from the viewpoint of expression efficiency in recombinant cells, solubility in water, and suppression of deliquescence.

More specifically, the protein of the present disclosure may be a protein set forth in SEQ ID NO: 1. This protein is a 571 amino acid long protein including 12 regions of about several tens of amino acids including Arg at position 745 to Asp at position 747 in SEQ ID NO: 2. Alternatively, the protein of the present disclosure may be a protein having the amino acid sequence set forth in SEQ ID NO: 3 or a protein having an amino acid sequence in which the amino acid sequence set forth in SEQ ID NO: 3 is repeated a plurality of times (for example, 2, 3, 4, 5, or 6 times). In addition, the protein of the present disclosure may be a protein having an amino acid sequence having 90% or more (or 95% or more) sequence identity to any of these amino acid sequences, and in this case, it is preferable that each RGD motif in the original amino acid sequence is maintained. The amino acid sequence of SEQ ID NO: 3 corresponds to the region from the amino acid at position 4 to the amino acid at position 192 of the amino acid sequence of SEQ ID NO: 1.

The pearl culture material of the present disclosure includes a pearl nucleus coated with the protein of the present disclosure or a mantle piece coated with the protein of the present disclosure, or both. Thus, for example, an embodiment in which a pearl nucleus coated with the protein of the present disclosure is used with an uncoated mantle piece, and an embodiment in which a mantle piece coated with the protein of the present disclosure is used with an uncoated pearl nucleus are also included in the scope of the present disclosure.

In a case where the pearl nucleus is coated with the protein of the present disclosure, it is sufficient that at least a part of the surface of the pearl nucleus is coated. On the surface of the pearl nucleus coated with the protein of the present disclosure, the percentage of the area coated with the protein of the present disclosure to the total surface area of the pearl nucleus is preferably 20% or more, more preferably 30% or more, still more preferably 40% or more, even more preferably 50% or more, even still more preferably 60% or more, and particularly preferably 65% or more. The entire surface of the pearl nucleus may be coated with the protein of the present disclosure. The amount of the protein of the present disclosure coated on the pearl nucleus surface is not particularly limited, but may be, for example, 0.001 g/m² to 100 g/m², and more specifically, 0.05 g/m² to 30 g/m².

Similarly, in a case where the mantle piece is coated with the protein of the present disclosure, it is sufficient that at least a part of the surface of the mantle piece is coated. On the surface of the mantle piece coated with the protein of the present disclosure, the percentage of the area coated with the protein of the present disclosure to the total surface area of the mantle piece is preferably 20% or more, more preferably 30% or more, still more preferably 40% or more, even more preferably 50% or more, even still more preferably 60% or more, and particularly preferably 65% or more. The entire surface of the mantle piece may be coated with the protein of the present disclosure. The amount of the protein of the present disclosure coated on the surface of the mantle piece is not particularly limited, but may be, for example, 0.001 g/m² to 100 g/m², and more specifically, 0.05 g/m² to 30 g/m².

In order to coat the pearl nucleus with the protein of the present disclosure, the protein of the present disclosure may be added to water and stirred to prepare a coating liquid, and the pearl nucleus may be coated with the coating liquid using a coating method known in the art. Examples of the coating method include a method of immersing the pearl nucleus in the coating liquid, a method of spraying the coating liquid on the pearl nucleus, and a method of coating the pearl nucleus with the coating liquid by a brush. The concentration of the protein of the present disclosure in the coating liquid is, for example, 0.0001% by mass to 1% by mass, and may be 0.0005% by mass to 0.5% by mass.

A salt such as NaCl or a buffer such as HEPES or PBS may coexist in the coating liquid, but it is preferable that the coating liquid is free of a substance harmful to the living body such as an organic solvent, from the viewpoint of avoiding adverse effects on the pearl oyster. The coating liquid may be a liquid coating composition itself among the coating compositions described later, or may be a solid coating composition or a liquid prepared by dissolving a freeze-dried product described later in a dissolution solution described later.

In order to coat the mantle piece with the protein of the present disclosure, the protein of the present disclosure may be added to water and stirred to prepare a coating liquid, and the mantle piece may be coated with the coating liquid using a coating method known in the art. Alternatively, the coating liquid may be coated on the mantle which may be then cut to prepare a mantle piece. Examples of the coating method include a method of immersing the mantle piece or the mantle in the coating liquid, a method of spraying the coating liquid on the mantle piece or the mantle, and a method of coating the mantle piece or the mantle with the coating liquid by a brush. The concentration of the protein of the present disclosure in the coating liquid is, for example, 0.0001% by mass to 1% by mass, and may be 0.0005% by mass to 0.5% by mass.

In a case of coating the mantle piece, a salt such as NaCl or a buffer such as HEPES or PBS may coexist in the coating liquid, but it is preferable that the coating liquid is free of a substance harmful to the living body such as an organic solvent, from the viewpoint of avoiding adverse effects on the pearl oyster. The coating liquid may be a liquid coating composition itself among the coating compositions described later, or may be a solid coating composition or a liquid prepared by dissolving a freeze-dried product described later in a dissolution solution described later.

<Coating Composition>

In accordance with the foregoing, the present disclosure also provides a coating composition for coating at least one selected from a pearl nucleus or a mantle piece. The coating composition contains a protein of the present disclosure. The coating composition preferably further contains at least one selected from an excipient, a carrier, or the like that is suitable and does not adversely affect a mother pearl oyster. The coating composition may be liquid or solid. Examples of the suitable liquid carrier include water, an alcohol, an animal or vegetable oil such as soybean oil or olive oil, a mineral oil, and a synthetic oil. Examples of the suitable solid carrier include a sugar such as maltose or sucrose, an amino acid, a cellulose derivative such as hydroxypropyl cellulose, and an organic acid salt such as magnesium stearate. A gel-like substance or the like can be used as the carrier.

In a case where the coating composition is solid, the composition may be dissolved in a suitable dissolution solution and then used to coat at least one selected from a pearl nucleus or a mantle piece. For example, physiological saline, a variety of buffers, a solution of a saccharide such as glucose, inositol, mannitol, or lactose, or a glycol such as ethylene glycol or polyethylene glycol is preferable as the dissolution solution. In addition, the coating composition may be a freeze-dried product obtained by freeze-drying the protein of the present disclosure together with an activation agent, for example, a sugar such as inositol, mannitol, lactose, sucrose, or trehalose or an amino acid such as phenylalanine. The freeze-dried product may be dissolved in a suitable dissolution solution, for example, a liquid such as sterilized water, physiological saline, glucose solution, electrolyte solution, or amino acid solution at the time of use and then used for coating.

The coating composition may further contain one or more selected from a salt such as NaCl, a buffer such as HEPES or PBS, a thickener, a pH adjuster, a stabilizer, a light absorber, and the like. In addition, the coating composition may contain an antibiotic such as a penicillin-based antibiotic, a cephem-based antibiotic, a macrolide-based antibiotic, a tetracycline-based antibiotic, or a new quinolone-based antibiotic. The coating composition may contain a colorant, such as phenol red or eosin. The concentration of the protein of the present disclosure in the coating composition is, for example, 0.0001% by mass to 1% by mass, and may be 0.0005% by mass to 0.5% by mass.

Use of the coating composition of the present disclosure makes it possible to produce at least one selected from a pearl nucleus or a mantle piece, which improves the yield of a high quality pearl and is excellent in the operability of nucleus insertion into a mother pearl oyster.

### EXAMPLES

Hereinafter, the present disclosure will be described more specifically with reference to Examples, but the present invention is not limited to the following Examples as long as the gist of the present invention is not exceeded. Unless otherwise specified, “parts” and “%” are based on mass.

### Examples 1 to 3

(Measurement of Weight-Average Molecular Weight (Mw), Number-Average Molecular Weight (Mn), and Polydispersity (Mw/Mn) by Gel Permeation Chromatography (GPC))

The weight-average molecular weight (Mw), number-average molecular weight (Mn), polydispersity (Mw/Mn), and viscosity of a cellnest human type I collagen-like recombinant peptide (manufactured by Fujifilm Corporation) were measured according to the following methods.

Here, the cellnest human type I collagen-like recombinant peptide has a structure in which a site containing an RGD sequence having high cell adhesiveness is cut out from human type I collagen α1 chain into four patterns of fragments which are then linked to each other, and the resulting three linked structures of four patterns of fragments are further linked. More specifically, the cellnest human type I collagen-like recombinant peptide has the amino acid sequence set forth in SEQ ID NO: 1, is made up of 571 amino acids in which the GXY triplet is repeated, and contains 12 RGD motifs.

The molecular weight of the cellnest human type I collagen-like recombinant peptide (manufactured by Fujifilm Corporation) was determined as a pullulan-converted molecular weight by the measurement using a GPC apparatus (HLC-8220GPC, manufactured by Tosoh Corporation) under the following conditions. An aqueous solution of a protein to be measured (after thawing for a frozen preparation) was heated at 40° C. for 30 minutes to completely dissolve the protein, diluted with 100 mM phosphate buffer such that the concentration of the protein to be measured was 0.2% by mass, and then filtered through a 0.45 μm filter to prepare a sample solution. The weight-average molecular weight and the number-average molecular weight of the protein to be measured were determined by measuring the sample solution using the above GPC apparatus, and the polydispersity was further calculated from the obtained weight-average molecular weight and number-average molecular weight.

[GPC Measurement Conditions]

Column: Shodex Asahipak GS-620 7G-P, inner diameter: 7.5 mm, length: 50 cm (manufactured by Showa Denko K.K.)

Eluent: 100 mM phosphate buffer (pH=6.9)

Flow rate: sample: 1.0 mL/min, reference: 0.1 mL/min

Column temperature (setting): 40° C.

Injection volume: 100 μL

Detector: RI/UV (210 nm)

Protein for molecular weight calibration: pullulan (Shodex P-82, manufactured by Showa Denko K.K.)

Table 1 shows the weight-average molecular weight (Mw), number-average molecular weight (Mn), and polydispersity (Mw/Mn) of the cellnest human type I collagen-like recombinant peptide determined as described above.

(Production of Coated Nucleus 1)

Water for injection (manufactured by Hikari Pharmaceutical Co., Ltd.) was added to 100 mg of the cellnest human type I collagen-like recombinant peptide (manufactured by Fujifilm Corporation, freeze-dried product), followed by mixing using a 300 ml Erlenmeyer flask (manufactured by AGC Techno Glass Co., Ltd.) to prepare a 0.1% coating aqueous solution 1. The cellnest human type I collagen-like recombinant peptide has the amino acid sequence of SEQ ID NO: 1.

An untreated nucleus (pearl nuclei) having a size of about 7 mm in diameter, which had been washed and sterilized, was completely immersed in the prepared coating aqueous solution 1 which was then shaken and stirred at 37° C. for 2 hours. Then, the mixture was filtered through a metal colander (16 mesh), and then dried under an atmosphere of a temperature of 25° C. and a humidity of 55% for 24 hours to produce a coated nucleus 1.

(Production of Coated Mantle Piece 1)

An untreated, common pearl-forming mantle piece was completely immersed in the coating aqueous solution 1 prepared as described above at 25° C. for 1 hour, and was taken out to prepare a coated mantle piece 1.

(Pearl Culture Experiment)

A group of 200 Pinctada fucata martensii mother pearl oysters (natural shellfishes, 2 years old) were prepared, and the nucleus insertion culture was carried out using coated nuclei 1 produced as described above and uncoated mantle pieces (Example 1), or coated mantles 1 and uncoated pearl nuclei (Example 2), and coated nuclei 1 and coated mantle pieces 1 (Example 3). The operability at the time of nucleus insertion, and the good quality pearl yield after extracting the nucleus from the shellfish eight months later, and the operability at the time of nucleus insertion were evaluated as follows.

—Good Quality Pearl Yield—

The presence or absence of a stain on the pearl surface and the area of the stain greatly affect the value of the pearl.

The good quality pearl yield was evaluated by using a percentage at which the stain area on the pearl surface was 0% or more and less than 5% (level of no stain to level at which slight stain was observed) as an evaluation index. Here, the stain is a generic term for abnormally deposited proteins that exist between the nucleus and the pearl layer or between the pearl layer and the pearl layer. The percentage of a brown to black colored area to a total surface area of the pearl was evaluated as the stain area. Specifically, the pearl was imaged with a digital camera from four directions, up, down, left, and right, and the stain area was calculated.

According to this evaluation index, the good quality pearl yield was determined according to the following evaluation standards. Note that the ratings of A and B are practically acceptable levels.

—Evaluation Standards—

A: The percentage of the area of the stain being 0% or more and less than 5% is 40% or more.

B: The percentage of the area of the stain being 0% or more and less than 5% is 35% or more and less than 40%.

C: The percentage of the area of the stain being 0% or more and less than 5% is 30% or more and less than 35%.

D: The percentage of the area of the stain being 0% or more and less than 5% is 25% or more and less than 30%.

E: The percentage of the area of the stain being 0% or more and less than 5% is less than 25%.

—Operability at the Time of Nucleus Insertion—

The efficiency of the operation of inserting a nucleus with an inserter at the time of nucleus insertion greatly affects the productivity of pearl culture.

The operability at the time of nucleus insertion was evaluated by a skilled operator of the nucleus insertion operation as to whether or not the operability of the inserter was good due to the tackiness (stickiness) of the nucleus at the time of nucleus insertion.

According to this evaluation index, the operability at the time of nucleus insertion was determined according to the following evaluation standards. Note that the rating of A is a practically acceptable level.

—Evaluation standards—

A: The operability was equivalent to the untreated nuclei.

B: The slight stickiness was felt as compared to the untreated nuclei, and therefore the operability was poor.

C: The clear stickiness was felt as compared to the untreated nuclei, and therefore the operability was extremely poor.

The evaluation results are shown in Table 1.

### Comparative Example 1

The good quality pearl yield and the operability at the time of nucleus insertion were evaluated in the same manner as in “Examples 1 to 3”, except that uncoated pearl nuclei and mantle pieces were used.

The evaluation results are shown in Table 1.

### Comparative Example 2 to Comparative Example 4

The weight-average molecular weight (Mw), number-average molecular weight (Mn), and polydispersity (Mw/Mn) were measured to evaluate the good quality pearl yield and the operability at the time of nucleus insertion, in the same manner as in “Examples 1 to 3”, except that the cellnest human type I collagen-like recombinant peptide was replaced with pig skin-derived type I collagen-PSILK (manufactured by NH Foods Ltd., pig skin collagen I solution).

The evaluation results are shown in Table 1.

### Comparative Example 5 to Comparative Example 7

The weight-average molecular weight (Mw), number-average molecular weight (Mn), and polydispersity (Mw/Mn) were measured to evaluate the good quality pearl yield and the operability at the time of nucleus insertion, in the same manner as in “Examples 1 to 3”, except that the cellnest human type I collagen-like recombinant peptide was replaced with high-grade gelatin-APAT (manufactured by Nippi, Inc.).

The evaluation results are shown in Table 1.

### Comparative Example 8 to Comparative Example 10

The weight-average molecular weight (Mw), number-average molecular weight (Mn), and polydispersity (Mw/Mn) were measured to evaluate the good quality pearl yield and the operability at the time of nucleus insertion, in the same manner as in “Examples 1 to 3”, except that the cellnest human type I collagen-like recombinant peptide was replaced with pig-derived collagen (manufactured by Nitta Gelatin Inc.).

The evaluation results are shown in Table 1.

### Comparative Example 11 to Comparative Example 13

The weight-average molecular weight (Mw), number-average molecular weight (Mn), and polydispersity (Mw/Mn) were measured to evaluate the good quality pearl yield and the operability at the time of nucleus insertion, in the same manner as in “Examples 1 to 3”, except that the cellnest human type I collagen-like recombinant peptide was replaced with pig-derived gelatin (manufactured by Nitta Gelatin Inc.).

The evaluation results are shown in Table 1.

### Comparative Example 14 to Comparative Example 16

The weight-average molecular weight (Mw), number-average molecular weight (Mn), and polydispersity (Mw/Mn) were measured to evaluate the good quality pearl yield and the operability at the time of nucleus insertion, in the same manner as in “Examples 1 to 3”, except that the cellnest human type I collagen-like recombinant peptide was replaced with bovine-derived gelatin (manufactured by Nitta Gelatin Inc.).

The evaluation results are shown in Table 1.

TABLE 1

| | | | | | | | Stain area | | | |
|---|---|---|---|---|---|---|---|---|---|---|
| | | | | Weight- | Number- | | in pearls [%] | | | |
| | | | | average | average | | 0% or | | Good | Operability |
| | Coated or uncoated | | | molecular | molecular | Poly- | more and | 5% | quality | at the time |
| | | Mantle | | weight | weight | dispersity | less than | or | pearl | of nucleus |
| | Pearl nucleus | piece | Coating material | (Mw) [Da] | (Mn) [Da] | (Mw/Mn) | 5% | more | yield | insertion |
| Example 1 | Coated | Uncoated | Cellnest human | 56494 | 48787 | 1.16 | 42 | 58 | A | A |
| Example 2 | Uncoated | Coated | type I collagen-like | | | | 41 | 59 | A | — |
| Example 3 | Coated | Coated | recombinant peptide | | | | 45 | 55 | A | A |
| | | | (manufactured by | | | | | | | |
| | | | Fujifilm Corporation) | | | | | | | |
| Comparative | Coated | Uncoated | Pig skin-derived type I | 226194 | 786 | 287.78 | 31 | 69 | C | C |
| Example 2 | | | collagen-PSILK | | | | | | | |
| Comparative | Uncoated | Coated | (manufactured by | | | | 31 | 69 | C | — |
| Example 3 | | | NH Foods Ltd.) | | | | | | | |
| Comparative | Coated | Coated | | | | | 34 | 66 | C | C |
| Example 4 | | | | | | | | | | |
| Comparative | Coated | Uncoated | High-grade gelatin- | 44342 | 464 | 95.56 | 30 | 70 | C | B |
| Example 5 | | | APAT (manufactured | | | | | | | |
| Comparative | Uncoated | Coated | by Nippi, Inc.) | | | | 31 | 69 | C | — |
| Example 6 | | | | | | | | | | |
| Comparative | Coated | Coated | | | | | 32 | 68 | C | B |
| Example 7 | | | | | | | | | | |
| Comparative | Coated | Uncoated | Pig-derived collagen | 121646 | 56 | 2172.25 | 34 | 66 | C | B |
| Example 8 | | | (manufactured by | | | | | | | |
| Comparative | Uncoated | Coated | Nitta Gelatin Inc.) | | | | 32 | 68 | C | — |
| Example 9 | | | | | | | | | | |
| Comparative | Coated | Coated | | | | | 34 | 66 | C | B |
| Example 10 | | | | | | | | | | |
| Comparative | Coated | Uncoated | Pig-derived gelatin | 227094 | 8135 | 27.92 | 35 | 65 | B | C |
| Example 11 | | | (manufactured by | | | | | | | |
| Comparative | Uncoated | Coated | Nitta Gelatin Inc.) | | | | 32 | 68 | C | — |
| Example 12 | | | | | | | | | | |
| Comparative | Coated | Coated | | | | | 35 | 65 | B | C |
| Example 13 | | | | | | | | | | |
| Comparative | Coated | Uncoated | Bovine-derived gelatin | 261944 | 4114 | 63.67 | 32 | 68 | C | B |
| Example 14 | | | (manufactured by | | | | | | | |
| Comparative | Uncoated | Coated | Nitta Gelatin Inc.) | | | | 31 | 69 | C | — |
| Example 15 | | | | | | | | | | |
| Comparative | Coated | Coated | | | | | 33 | 67 | C | B |
| Example 16 | | | | | | | | | | |
| Comparative | Uncoated | Uncoated | None | — | — | — | 23 | 77 | E | A |
| Example 1 | | | | | | | | | | |

It was shown that the good quality pearl yield and the operability at the time of nucleus insertion were excellent in a case where the cellnest human type I collagen-like recombinant peptide having the amino acid sequence set forth in SEQ ID NO: 1, made up of 571 amino acids in which GXY triplets are repeated, containing 12 RGD motifs, and having a polydispersity of 1.16 was coated on the pearl nucleus, the mantle piece, or both the pearl nucleus and the mantle piece (Examples 1 to 3).

On the other hand, it was shown in Comparative Examples 1 to 16 that at least one of the good quality pearl yield or the operability at the time of nucleus insertion was at an evaluation level that was not practically acceptable.

The disclosure of JP2017-214180 filed on Nov. 6, 2017 is incorporated herein by reference in its entirety.

All publications, patent applications, and technical standards mentioned in this specification are herein incorporated by reference to the same extent as if each individual publication, patent application, and technical standard were specifically and individually indicated to be incorporated by reference.

The pearl culture material of the present disclosure can be preferably used for producing a good quality pearl.

What is claimed is:

1. A pearl culture material in which at least one selected from the group consisting of a pearl nucleus and a mantle piece is coated with a protein containing an amino acid sequence set forth in SEQ ID NO: 3, and having a polydispersity of less than 20.

2. The pearl culture material according to claim 1,
wherein the protein has a weight-average molecular weight of 30 kDa to 200 kDa determined by gel permeation chromatography.

3. The pearl culture material according to claim 1,
wherein the protein comprises at least a part of an amino acid sequence of collagen.

4. The pearl culture material according to claim 3,
wherein the amino acid sequence of the collagen is an amino acid sequence of type I collagen α1 chain.

5. A coating composition for coating at least one selected from a pearl nucleus or a mantle piece, comprising:
a protein containing an amino acid sequence set forth in SEQ ID NO: 3, and having a polydispersity of less than 20.

6. The coating composition according to claim 5,
wherein the protein has a weight-average molecular weight of 30 kDa to 200 kDa determined by gel permeation chromatography.

7. The coating composition according to claim 5,
wherein the protein comprises at least a part of an amino acid sequence of collagen.

8. The coating composition according to claim 7,
wherein the amino acid sequence of the collagen is an amino acid sequence of type I collagen α1 chain.

9. The coating composition according to claim 5, wherein the coating composition further comprises at least one selected from the group consisting of an excipient and a carrier.
